(19) United States
(12) Patent Application Publication
(10) Pub. No.: US 2005/0154423 A1
(43) Pub. Date: Jul. 14, 2005

(54) Method for reducing implantable defibrillator volume

(76) Inventors: Steven D. Goedeke, Forest Lake, MN (US); Forrest C.M. Pape, New Brighton, MN (US)

Correspondence Address: MEDTRONIC, INC., 710 MEDTRONIC PARKWAY NE, MS-LC340, MINNEAPOLIS, MN, 55432-5604, US

(21) Appl. No.: 10/756,479

(22) Filed: Jan. 13, 2004

(51) Int. Cl. A61N 1/375

(52) U.S. Cl. 607/36

(57) ABSTRACT

A battery/capacitor assembly for use in an implantable medical device comprises a battery and at least one capacitor housed in a unitary metal encasement. Either the battery, capacitor, or both may be coated with an insulating layer (e.g. plastic). In addition, a metallic layer may be disposed between the battery and the at least one capacitor.

### TECHNICAL FIELD OF THE INVENTION

The present invention generally relates to implantable medical devices, and more particularly to a method and apparatus for reducing the volume of an implantable medical device.

### BACKGROUND OF THE INVENTION

Implantable medical devices (IMDs) such as cardiac stimulators, neuro-stimulators, muscular stimulators, etc. are well known. While the present invention will be described in connection with implantable medical devices such as pacemakers or defibrillators, it should be understood that the principles herein may have applicability to other implantable medical devices as well.

An implantable medical device (IMD) such as an implantable pulse generator (IPG), commonly referred to as a pacemaker, may be used to stimulate the heart into a contraction when the associated rhythm of the heart is an abnormal rhythm. Modern cardiac devices also perform many other functions beyond that of pacing. For example, some cardiac devices such as implantable cardioverter defibrillators (IMD) may also perform therapies such as defibrillation and cardioversion as well as providing several different pacing therapies, depending upon the needs of the user or patient and the physiologic condition of the patient's heart. For convenience, all types of implantable medical devices will be referred to herein as IMDs, it being understood that the term, unless otherwise indicated, is inclusive of an implantable device capable of administering any one of a number of therapies to the heart of a patient.

Typically, an IMD is implanted in a convenient location usually under the skin of a patient in the vicinity of the one or more major arteries or veins. One (or more) electrical leads connected to the IMD is inserted into or deployed on the heart of the user, usually through a convenient vein or artery. The ends of the leads are placed in contact with the walls or surface of one or more chambers of the heart, depending upon the particular therapy deemed appropriate for the patient.

One or more of the leads is adapted to carry a current from the IMD to the heart tissue to stimulate the heart in one of several ways, again depending upon the particular therapy being delivered. The leads are simultaneously used for sensing the physiologic signals provided by the heart to determine when to deliver a therapeutic pulse to the heart, and the nature of the pulse; e.g., a pacing pulse or a defibrillation shock. Such IMDs are typically housed in a container or can that is made Of metal or some other conductive material. The can is made of conductive material because in some circumstances the can itself is used as one of the electrodes for sensing the physiologic indicia of the patient.

In IMDs that deliver defibrillation or cardioversion therapies, it is necessary to develop high voltages, perhaps 750 volts or more, within the IMD in order to administer a sufficient shock to a patient to correct an arrhythmia or a fibrillation, particularly a ventricular fibrillation. To generate such high voltages, a large battery and a large capacitor (usually, two capacitors) may be used. Typically the battery is encased in a first metal container within the IMD, and the capacitors are encased within a separate metal container. Thus, there are at least two layers of metal between the battery and the capacitor(s), adding to the volume of the IMD. Additionally, to facilitate fabrication and assembly, the separately metal-encased battery and capacitor(s) may be inserted into a cradle (e.g. plastic) having separate battery and capacitor positioning compartments. Thus, in addition to the two layers of metal, there may also be a plastic region separating the battery and capacitor(s), further increasing the volume of the IMD. Volume is a major consideration in the design of implantable medical devices since the device must be placed within a patient's body, and a large device may be more difficult to implant and/or more uncomfortable to the user. However, because the form factor of the batteries and capacitors currently in use are dissimilar and non-compatible when packaged together, the problem of volume retention is somewhat difficult to address.

Accordingly, it is desirable to provide a method and apparatus for reducing the volume of an implantable medical device. In addition, it is desirable to modify the form function of a battery and capacitor for use in an implantable medical device. Furthermore, other desirable features and characteristics of the present invention will become apparent from the subsequent detailed description and the appended claims, taken in conjunction with the accompanying drawings and the foregoing technical field and background.

### BRIEF SUMMARY OF THE INVENTION

According to an aspect of the invention, there is provided a battery/capacitor assembly for use in an implantable medical device. The assembly comprises a battery, at least one capacitor, and a unitary metal encasement for retaining the battery and the at least one capacitor in proximity.

According to a further aspect of the invention, there is provided a method for reducing the volume of an implantable medical device of the type that utilizes a battery and at least one capacitor. The battery and the at least one capacitor are encased in a unitary metal housing.

According to a still further aspect of the invention, there is provided a battery/capacitor assembly for use in an implantable medical device. The assembly comprises a battery, a capacitor, a unitary metal encasement for retaining the battery and the capacitor in proximity, and an electrically insulating layer disposed around one of the battery and the capacitor.

### BRIEF DESCRIPTION OF THE DRAWINGS

The present invention will hereinafter be described in conjunction with the following drawing figures, wherein like numerals denote like elements, and

FIG. 1 is a diagram showing a typical placement of an IMD in a user;

FIG. 2 is an isometric view of a typical IMD battery and capacitor assembly in accordance with the prior art;

FIG. 3 is a top view of the battery/capacitor assembly shown in FIG. 2.

FIG. 4 is a cross-sectional view of the battery/capacitor assembly shown in FIG. 3 taken along ling 4-4;

FIG. 5 is an isometric view of an improved battery/capacitor assembly for use in an IMD;

FIG. 6 is an isometric view of another improved battery/capacitor assembly for use in an IMD;

FIG. 7 is a schematic diagram of a defibrillator capacitor charging circuit;

FIG. 8 is a cross-sectional view of a first embodiment of an inventive battery/capacitor assembly for use in an IMD;

FIG. 9 is a cross-sectional view of a further embodiment of the present invention;

FIG. 10 is a cross-sectional view of a still further embodiment of the present invention;

FIG. 11 is a cross-sectional view of yet a further embodiment of the present invention;

FIG. 12 is a cross-sectional view of yet a further embodiment of the present invention; and

FIG. 13 is a cross-sectional view of a still further embodiment of the present invention.

### DETAILED DESCRIPTION OF THE INVENTION

The following detailed description is merely exemplary in nature and is not intended to limit the invention or the application and uses of the invention. Furthermore, there is no intention to be bound by any expressed or implied theory presented in the preceding technical field, background, brief summary or the following detailed description.

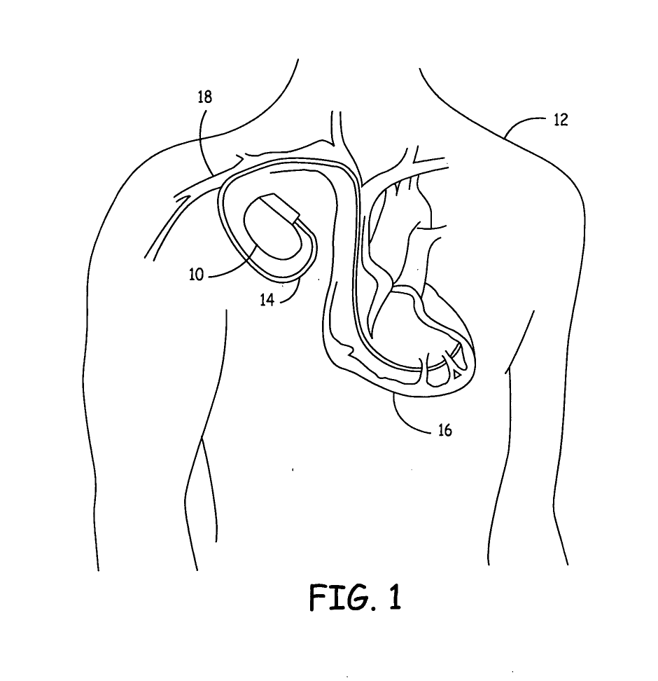

FIG. 1 is an illustration showing generally where an implantable cardiac device (IMD) is placed in a conventional manner in a patient 12. The IMD is conventionally housed within a hermetically sealed, biologically inert outer canister or housing 10, which itself may be of a conductive material and serve as an electrode in the IMDs pacing/sensing circuit. One or more leads, collectively identified as 14 are electrically coupled to the IMD in a conventional manner, extending within a patient 16, such as within a heart 16 of the patient 16 via a vein 18. Disposed generally near the distal end of lead 14 are one or more exposed conductive electrodes for receiving electrical cardiac signals and/or for delivering electrical stimuli or other therapies to heart 16. Lead 14 may be implanted with its distal end in either the atrium or the ventricle of heart 16. Lead 14 is preferably a bipolar lead such that lead 14 actually has two separate and mutually insulated leads, the first having a terminal at the distal end of lead 14 and the second having a terminal near, but set back from the distal end. Such leads are well known in the art.

An implantable cardiac device (IMD) according to an embodiment of the present invention includes a pulse generator for producing pulses that are used to pace the heart; i.e., cause a depolarization of the heart tissue or issue a defibrillation pulse to shock the heart from arrhythmia to a normal heart beat. A processor within the IMD analyzes the sensed pulses to determine whether a therapy should be administered. As noted above, although the present invention may have applicability to a number of types of implantable medical devices and particularly to IMDs, the following description will utilize as exemplary an implantable cardiac device.

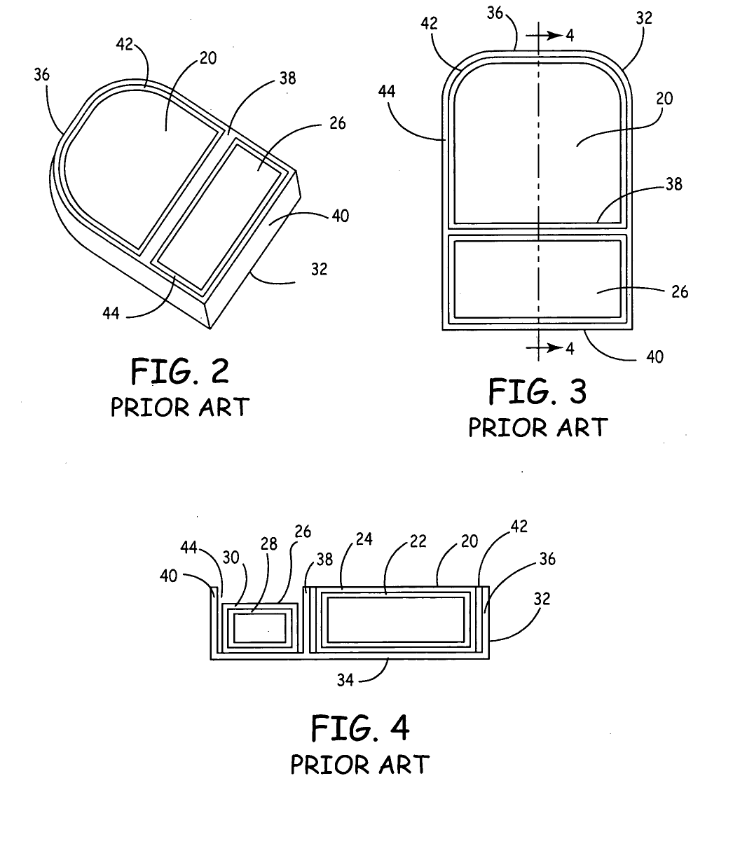

FIGS. 2, 3, and 4 are isometric, top, and cross-sectional views of a battery/capacitor assembly for use in an implantable medical device in accordance with the teachings of the prior art. A capacitor 20 includes a plastic coating 22, and the combination is enclosed within a metal canister 24. It should be clear that one or more capacitors may be employed where required. A battery 26 likewise includes a plastic layer 28, and the combination is enclosed in metal canister 30. To facilitate production and assembly, capacitor 20 and battery 26 are placed in separate compartments of a piece-part (e.g. plastic) 32 in the form of a cradle. Piece-part 32 includes a base 34 and walls 36, 38, and 40 which form separate compartments 42 and 44 to individually cradle capacitor 20 and battery 26 respectively. Associated circuit boards, connection wires, and other components of the IMD are not shown for clarity. As can be seen from FIGS. 2, 3, and 4 two layers of metal 24 and 30 and interior wall 38 of piece-part 32 separate capacitor 20 and battery 26. Since, as described above, it would be desirable to reduce the volume of IMDs in order to make IMDs easier to implant and more comfortable for the patient, it would be desirable to eliminate the above described two layers of metal and intermediate wall 38.

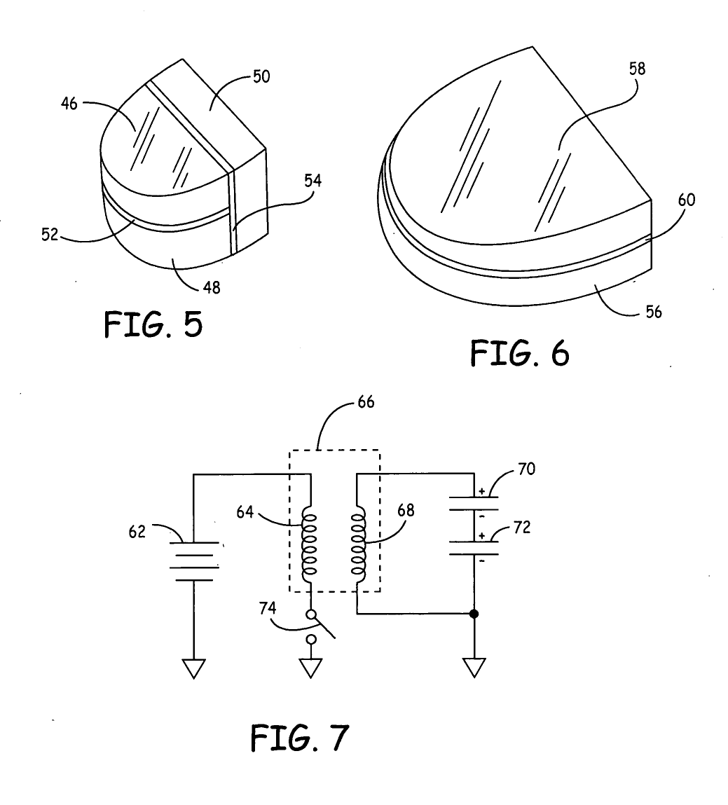

FIGS. 5 and 6 are isometric views of battery/capacitor assemblies having an improved configuration. Referring to FIG. 5, there are shown two stacked capacitors 46 and 48 and a battery 50. Instead of enclosing capacitors 46 and 48 and battery 50 in individual metal encasements, capacitors 46 and 48 are separated by a layer of insulating material 52 (e.g. plastic). In addition, battery 50 is separated from capacitors 46 and 48 by a second layer of insulating material 54 (e.g. plastic). It should be appreciated, that the volume of the IMD utilizing this battery/capacitor assembly is substantially reduced by eliminating the layers of metal separately encasing the capacitors and the battery, as was the case with the prior art assemblies shown in FIGS. 2-4.

Referring to FIG. 6, there is shown a battery 56 and a capacitor 58 both having the same general form factor. It should be appreciated, however, that one may be thicker than the other. A layer of insulating material 60 is placed between battery 56 and capacitor 58 to provide electrical isolation, if necessary. As will be described below, depending upon the design of battery 56 and capacitor 58, insulating layer 60 may not be necessary and may be replaced by a thin conductive layer which may serve as a common terminal for battery 56 and capacitor 58. The conductive layer may also provide chemical isolation between battery 56 and capacitor 58 if such is required. As was the case with the battery/capacitor assembly shown on FIG. 6, more than one capacitor may be employed. If, for example, two capacitors are used and stacked upon one another, an insulating layer may be required between the stacked capacitors.

FIG. 7 is a schematic diagram of a typical defibrillator capacitor charging circuit. A battery 62 is coupled in parallel with the primary winding 64 of a charging coil or step-up transformer 66. The secondary winding 68 of step-up transformer 66 is coupled in parallel with two series capacitors 70 and 72. Switch 74 is coupled in series with primary winding 64 and is utilized to interrupt the flow of current from battery 62 through primary winding 64, which current is used to charge capacitors 70 and 72 in anticipation of administering a therapy shock to a patient. A separate disk charge circuit (not shown) discharges capacitors 70 and 72 through a lead of the IMD to the patient.

As can be seen, the negative terminal of battery 64 and the negative side of capacitor 72 are at the same electrical potential. Depending on the design of battery 62 and capacitor 72, it is possible to eliminate the thin layer of electrically isolating material referred to above between battery 62 and capacitor 72 thus saving volume. Alternatively, the isolating layer may be replaced by a thin conductive layer. There are several options to the solution of providing electrical and chemical isolation and the amount of chemical isolation required between the battery and the capacitors. As noted above, if a circuit is designed such that the battery and capacitor have a terminal at the same electrical potential, then isolation is required only to keep the battery and capacitors chemically separate. If necessary, both electrical and chemical isolation may be provided as described more fully below.

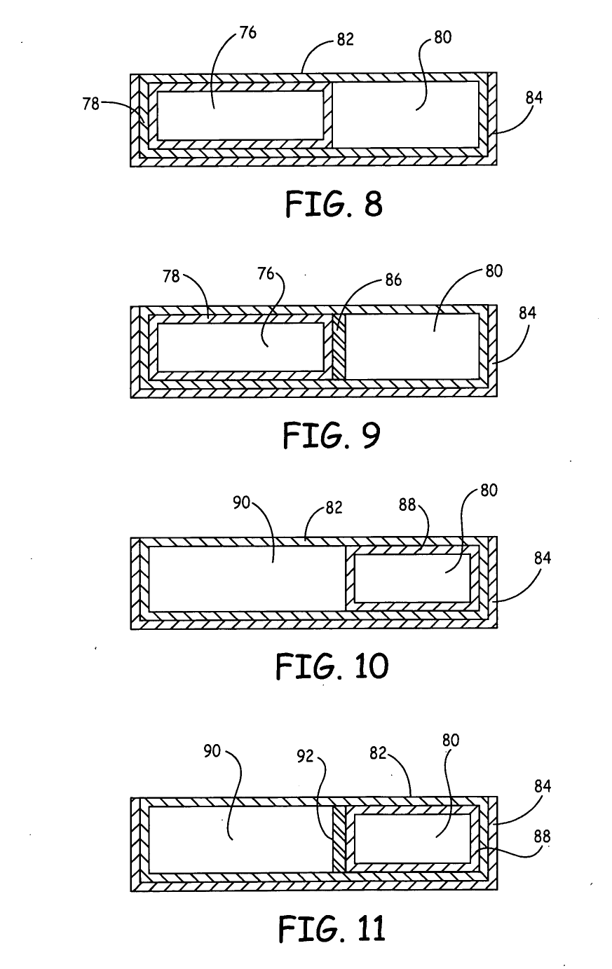

FIG. 8 is a cross-sectional view of a first embodiment of the inventive battery/capacitor assembly for use in an IMD according to the present invention. Referring to FIG. 8, a capacitor 76 having a layer 78 of plastic coating or wrapping thereon is placed in side-by-side abutment with battery 80. Both plastic coated capacitor 76 and battery 80 are housed in a single unitary metallic canister 82. While the battery and capacitor shown in FIG. 8 are positioned in a side-by-side relationship, it should be understood that other deployments of the battery and capacitor are contemplated by the invention. For example, battery 80 could be deployed on the top of or bottom of capacitor 76 and still be housed in a single unitary metallic canister. If desired, canister 82 containing capacitor 76 and battery 80 may be then be placed in a cradle 84 (e.g. plastic), as was previously described.

The layer of insulating material 78 electrically isolates capacitor 76 from battery 80. Additional isolation (e.g. chemical isolation) may be provided by placing a metallic wall 86 (FIG. 9) between capacitor 76 and battery 80. In addition to enhancing isolation between capacitor 76 and battery 80, metal wall 86 will enhance the rigidity of the battery/capacitor assembly and may also serve as a common terminal for battery 80 and capacitor 76 should each of these components have a terminal at the same potential as described above in connection with FIG. 7.

FIG. 10 illustrates an alternative embodiment of the present invention wherein battery 80 is provided with a layer of insulative coating or wrapping 88 (e.g. plastic). Battery 80, wrapped in insulative coating 88, is placed in side-by-side abutment with a capacitor 90 as was the case previously. It should be appreciated by those skilled in the art that other arrangements between battery 80 and capacitor 90 may be utilized and still housed within a single metallic canister 82. As was discussed previously, canister 82 may then be placed in a cradle 84 for handling and assembly. A further isolation wall (e.g. conductive wall 92) may be placed between battery 80 and capacitor 90 to serve as a strength member and/or provide chemical isolation as is shown in FIG. 11.

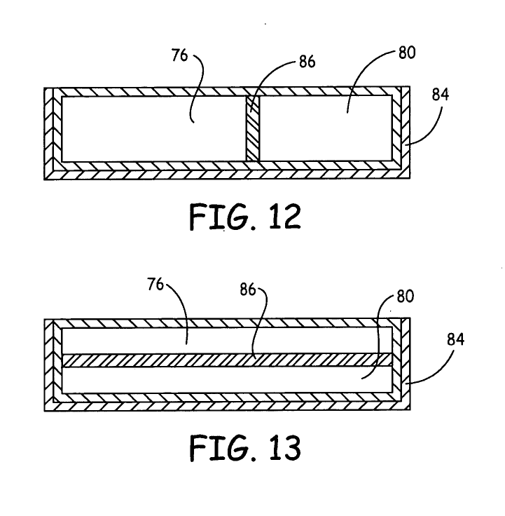

FIG. 12 illustrates yet another embodiment of the present invention wherein capacitor 76 and battery 80 are housed in a single unitary canister 82 and are separated by metallic wall 86. In FIG. 13, capacitor 76 and battery 80 are stacked and separated by metallic wall 86.

Thus, there has been provided, a battery/capacitor assembly for use in an implantable medical device wherein the volume of the battery/capacitor assembly has been significantly reduced. That is, it has been shown how separate metallic canister walls between the battery and the capacitor can be eliminated by employing a single unitary metallic canister which houses both the battery and capacitor. Furthermore, the intermediate cradle wall utilized in the prior art has been eliminated producing additional volume savings. The embodiments in the present invention described above illustrate how both electrical and/or chemical isolation may be provided. While certain of the embodiments include the addition of a metallic separator between the battery and capacitor, the width of this wall is less than the combined width of the dual metallic canister walls employed in the prior art.

1. A battery/capacitor assembly for use in an implantable medical device, said assembly comprising:
a battery;
at least one capacitor; and
a unitary metal encasement for retaining said battery and said at least one capacitor in proximity.

2. An assembly according to claim 1 further comprising a layer of isolation material interposed between said battery and said at least one capacitor.

3. An assembly according to claim 2 wherein said isolation material is a chemical isolation material.

4. An assembly according to claim 3 wherein said chemical isolation material is metallic.

5. An assembly according to claim 2 further comprising an electrically isolating material disposed around said battery.

6. An assembly according to claim 5 wherein said isolating material is an insulator.

7. An assembly according to claim 6 wherein said insulator is plastic.

8. An assembly according to claim 2 further comprising an electrically isolating material disposed around said at least one capacitor.

9. An assembly according to claim 8 wherein said isolating material is an insulator.

10. An assembly according to claim 9 wherein said insulator is plastic.

11. An assembly according to claim 1 further comprising a electrically isolating material around one of said battery and said at least one capacitor.

12. An assembly according to claim 11 wherein said isolating material is an insulator.

13. An assembly according to claim 12 wherein said insulator is plastic.

14. An assembly according to claim 11 further comprising a layer of chemically isolating material interposed between said battery and said at least one capacitor.

15. An assembly according to claim 1 further comprising a cradle for receiving said unitary metal encasement.

16. An assembly according to claim 15 wherein said cradle is plastic.

17. An assembly according to claim 1 wherein said at least one capacitor comprises:
a first capacitor;
a second capacitor; and
a first layer of insulating material disposed between said first capacitor and said second capacitor.

18. An assembly according to claim 1 wherein said at least one capacitor comprises:
a first capacitor;
a second capacitor; and
a first layer of metal disposed between said first capacitor and said second capacitor.

19. An assembly according to claim 17 further comprising a second layer of insulating material disposed between said battery and said first and second capacitors.

20. An assembly according to claim 2 wherein said layer of isolation material provides a common terminal for said battery and said at least one capacitor.

21. An assembly according to claim 2 wherein said battery and said at least one capacitor have a complementary form to maximize an area of abutment between said battery and said at least one capacitor.

22. A method for reducing the volume of an implantable medical device of the type that utilizes a battery and at least one capacitor, the method comprising:
encasing said battery and said at least one capacitor in a unitary metal housing.

23. An assembly according to claim 22 further comprising interposing a layer of insulating material between said battery and said at least one capacitor to electrically isolate said battery and said at least one capacitor.

24. An assembly according to claim 22 further comprising interposing a layer of conducting material between said battery and said at least one capacitor to chemically isolate said battery and said at least one capacitor.

25. An assembly according to claim 23 further comprising interposing a layer of conducting material between said battery and said at least one capacitor to chemically isolate said battery and said at least one capacitor.

26. An assembly according to claim 22 further comprising interposing a layer of conductive material between said battery and said at least one battery to form a common electrical terminal of said battery and said at least one capacitor.

27. A battery/capacitor assembly for use in an implantable medical device, said assembly comprising:
a battery;
a capacitor;
a unitary metal encasement for retaining said battery and said capacitor in proximity; and
an electrically insulating layer disposed around one of said battery and said capacitor.

28. An assembly according to claim 27 further comprising a layer of chemically isolating material interposed between said battery and said capacitor.

29. An assembly according to claim 27 further comprising a reinforcing member interposed between said battery and said capacitor.

30. An assembly according to claim 28 wherein said insulating layer is plastic.

31. An assembly according to claim 28 wherein chemically isolating material is metallic.
